(19) United States
(12) Patent Application Publication
(10) Pub. No.: US 2017/0173022 A1
(43) Pub. Date: Jun. 22, 2017

(54) MGLU2/3 ANTAGONISTS FOR THE TREATMENT OF AUTISTIC DISORDERS

(71) Applicant: Hoffmann-La Roche Inc., Little Falls, NJ (US)

(72) Inventors: Silvia Gatti McArthur, Basel (CH); Michael Saxe, Basel (CH); Juergen Wichmann, Steinen (DE); Thomas Woltering, Freiburg (DE)

(73) Assignee: Hoffmann-La Roche Inc., Little Falls, NJ (US)

(21) Appl. No.: 15/365,535

(22) Filed: Nov. 30, 2016

Related U.S. Application Data
(63) Continuation of application No. 14/694,625, filed on Apr. 23, 2015, now abandoned.
Application No. 14/694,625 is a continuation of application No. PCT/EP2013/071921, filed on Oct. 21, 2013.

(30) Foreign Application Priority Data
Oct. 23, 2012 (EP) 12189553.6

(51) Int. Cl. A61K 31/519; A61K 9/48; A61K 9/00; C07D 401/04; A61K 9/16; A61K 9/02; A61K 47/10; C07D 487/04; A61K 31/444; A61K 9/20

(52) U.S. Cl. CPC A61K 31/519; A61K 31/444; A61K 9/4858; A61K 9/0031; A61K 9/0019; A61K 9/4866; A61K 9/2054; A61K 9/0095; A61K 9/2018; A61K 9/4825; A61K 9/1652; A61K 9/02; A61K 47/10; C07D 487/04; C07D 401/04; A61K 2121/00

(57) ABSTRACT

This invention relates to a new medical use for certain chemical compounds and pharmaceutical compositions containing them. The invention relates to compounds which are mGlu2/3 negative allosteric modulators for use in the treatment of Autistic Spectrum Disorder (ASD), in particular, autism. In another aspect, the invention relates to a pharmaceutical composition for use in the treatment of ASD comprising a compound according to the invention and a pharmaceutically acceptable carrier.

This application is a continuation of U.S. application Ser. No. 14/694,625, filed Apr. 23, 2015, which is a continuation of International Application PCT/EP2013/071921, filed Oct. 21, 2013, which claims the benefit of priority to European Application 12189553.6, filed Oct. 23, 2012, each of which is incorporated herein by reference in its entirety.

### SUMMARY

This invention relates to a new medical use for certain chemical compounds and pharmaceutical compositions containing them. The invention relates to compounds which are mGlu2/3 negative allosteric modulators for use in the treatment of ASD, in particular autism. In another aspect, the invention relates to a pharmaceutical composition for use in the treatment of ASD comprising a compound according to the invention and a pharmaceutically acceptable carrier.

### BACKGROUND ART

L-glutamic acid, the most commonly occurring neurotransmitter in the CNS, plays a critical role in a large number of physiological processes. The glutamate-dependent stimulus receptors are divided into two main groups. The first main group forms ligand-controlled ion channels. The metabotropic glutamate receptors (mGluR) form the second main group and, furthermore, belong to the family of G-protein-coupled receptors.

At present, eight different members of these mGluR are known and of these some even have sub-types. On the basis of structural parameters, the different influences on the synthesis of intracellular signaling molecules and the different affinity to low-molecular weight chemical compounds, these eight receptors can be sub-divided into three sub-groups: mGlu1 and mGlu5 belong to group I, mGlu2 and mGlu3 belong to group II and mGlu4, mGlu6, mGlu7 and mGlu8 belong to group III.

Ligands of metabotropic glutamate receptors belonging to the group II have been known for the treatment or prevention of acute and/or chronic neurological disorders such as psychosis, schizophrenia, major depression and Alzheimer's disease.

Preferred compounds for use according to the invention are those compounds which act as mGlu2/3 negative allosteric modulators are described in WO 01/29011¹, WO 01/29012², WO 02/083652³, WO 02/083665⁴, WO 03/066623⁵, WO 2005/014002⁶, WO 2005/040171⁷, WO 2005/123738⁸, WO 2006/084634⁹, WO 2006/099972¹⁰, WO 2007/039439¹¹, WO 2007/110337¹² and WO 2008/119689¹³.

Autistic Spectrum Disorders (ASD) are a clinically heterogeneous condition characterized by defects in socialization and language. ASD include a wide range of abnormalities including a genuine incapacity to organise affective relations, behavioural anomalies in reciprocal social interactions, verbal and non verbal communication, limited interest in the surrounding environment associated with stereotyped movements and repetitive plays (Bourreau et al, 2009)¹⁴. Research to date indicates that a genetic predisposition may be involved, but also environmental factors have to be taken into consideration (Bourgeron, 2009)¹⁵. There is at present no efficient biological/pharmaceutical treatment to ASD.

### BRIEF DESCRIPTION OF THE FIGURES

FIG. 1: Social behavior test box, where a mouse is given a choice between staying in the center chamber, spending time in the side chamber with an unfamiliar mouse (stimulus mouse), or spending time in the side chamber with an inanimate object during social preference tests. Stranger mice were enclosed in wire cages (cups).

FIG. 2a, FIG. 2b and FIG. 2c: 3-Chambered social test results (animal vs. object), duration in the chamber

FIG. 3a, FIG. 3b, and FIG. 3c: 3-Chambered social test results (animal vs. object), duration sniffing

FIG. 4a, FIG. 4b, FIG. 4c, FIG. 4d and FIG. 4e: Distribution and abundance of [³1-1]LY354740 binding to brain sections of mGlu2 BTBR mice

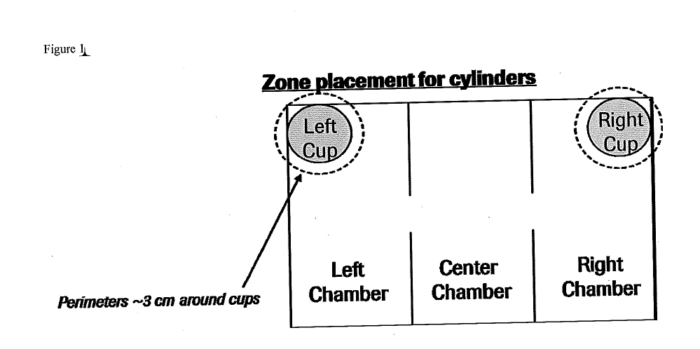

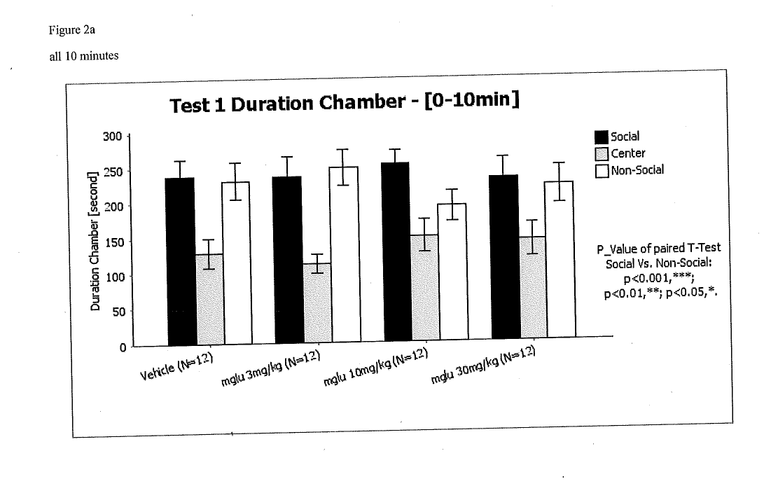

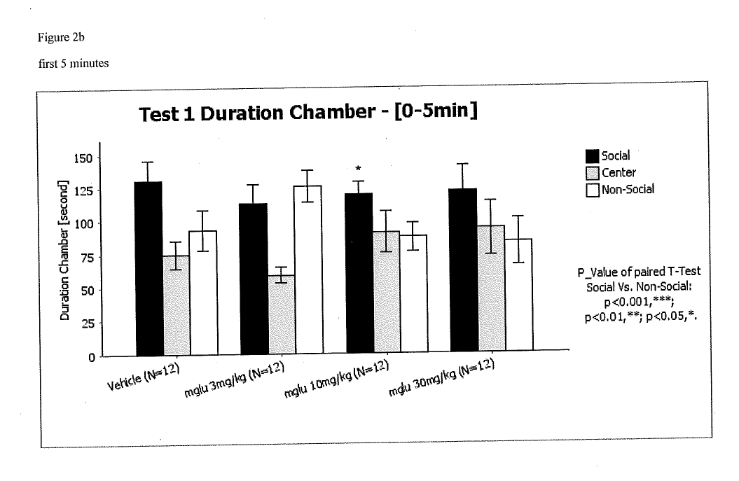

### DETAILED DESCRIPTION OF THE INVENTION

The terms “Autistic Spectrum” and “Autistic Spectrum Disorders” summarize conditions classified as pervasive developmental disorders, which include but are not limited to autism, Asperger syndrome, pervasive developmental disorder not otherwise specified (PDD-NOS), childhood disintegrative disorder, Rett syndrome and Fragile X, in particular autism. These disorders are typically characterized by social deficits, communication difficulties, stereotyped or repetitive behaviors and interests, and cognitive delays.

The following definitions of the general terms used in the present description apply irrespectively of whether the terms in question appear alone or in combination with other groups.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below.

The nomenclature used in this Application is based on IUPAC systematic nomenclature, unless indicated otherwise.

The term “modulator” denotes a molecule that interacts with a target receptor. The interactions include e.g. agonistic, antagonistic, or inverse agonistic activity.

The term “allosteric modulator” denotes a compound that binds to a receptor at a site distinct from the agonist binding site (an “allosteric site”). It induces a conformational change in the receptor, which alters the activation of the receptor when in presence of the endogenous ligand or agonist. “Positive allosteric modulators” increase the affinity and/or the activity of agonists, whilst “negative allosteric modulators” (NAM) decrease the activity and/or the affinity (and hence decrease the activity) of agonists for a receptor.

The term “C₁₋₆-alkyl”, alone or in combination with other groups, stands for a hydrocarbon radical which may be linear or branched, with single or multiple branching, wherein the alkyl group in general comprises 1 to 6 carbon atoms, for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl, 1,2-dimethyl-propyl and the like. Particular “C₁₋₆-alkyl” groups have 1 to 4 carbon atoms. A specific group is CH₃.

The terms “halogen-C₁₋₆-alkyl” or “C₁₋₆-haloalkyl”, alone or in combination with other groups, refers to C₁₋₆-alkyl as defined herein, which is substituted by one or multiple halogen, in particular 1-5 halogen, more particular 1-3 halogen (“halogen-C₁₋₃-alkyl”), specific groups have 1 halogen or 3 halogens. Particular halogen is fluoro (“fluoro-C₁₋₆-alkyl”) A particular “halogen-C₁₋₆-alkyl” group is fluoro-C₁₋₆-alkyl, more particular CF₃.

The term “C₂₋₆-alkenyl” denotes straight-chain or branched unsaturated hydrocarbon residues with 2 to 6 carbon atoms, preferably with 2 to 4 carbon atoms, such as ethenyl or propenyl.

The term “ C₂₋₆-alkoxy-(ethoxy),” (r is 1, 2, 3 or 4) denotes a lower alkoxy residue in the sense of the foregoing definition bound via 1 to 4 —CH₂—CH₂—O— groups, for example 2-methoxy-ethoxy.

The term “amino”, alone or in combination with other groups, refers to NH₂.

The term “cyano”, alone or in combination with other groups, refers to N≡C—(NC—).

The term “nitro”, alone or in combination with other groups, refers to NO₂.

The term “hydroxy”, alone or in combination with other groups, refers to —OH.

The terms “halogen” or “halo”, alone or in combination with other groups, denotes chloro (Cl), iodo (I), fluoro (F) and bromo (Br). Particular “halogen” is Cl and F. Specific is F

The term “aryl”, alone or in combination with other groups, refers to an aromatic carbocyclic group containing 6 to 14, in particular 6 to 10, carbon atoms and having at least one aromatic ring or multiple condensed rings in which at least one ring is aromatic. Examples of “aryl” include benzyl, biphenyl, indanyl, naphthyl, phenyl (Ph) and the like. Particular “aryl” is phenyl.

The term “heteroaryl”, alone or in combination with other groups, refers to an aromatic carbocyclic group of having a single 4 to 8 membered ring or multiple condensed rings containing 5 to 14, in particular 5 to 12 ring atoms and containing 1, 2 or 3 heteroatoms individually selected from N, O and S, in particular N and O, in which group at least one heterocyclic ring is aromatic. A “six-membered aromatic heterocycle” means a single aromatic ring containing 1-3 nitrogens or a pyridine-N-oxide. “Examples of “heteroaryl” include benzofuryl, benzoimidazolyl, 1H-b enzoimidazolyl, benzooxazinyl, benzoxazolyl, benzothiazinyl, benzothiazolyl, benzothienyl, benzotriazolyl, furyl, imidazolyl, indazolyl, 1H-indazolyl, indolyl, isoquinolinyl, isothiazolyl, isoxazolyl, oxazolyl, pyrazinyl, pyrazolyl (pyrazyl), 1H-pyrazolyl, pyrazolo[1,5-a]pyridinyl, pyridazinyl, pyridinyl, pyrimidinyl, pyrrolyl, quinolinyl, tetrazolyl, thiazolyl, thienyl, triazolyl, 6,7-dihydro-5H-[1]pyrindinyl and the like. Particular “heteroaryl” are pyridin-2-yl, pyridin-3-yl, pyridine-4-yl, pyrimidin-4-yl, pyrimidin-5-yl, pyridazin-2-yl, pyridazin-3-yl, thiazol-2-yl, thiazol-5-yl, and thiophen-2-yl.

The term “pyridine-N-oxide” or “pyridine-1-oxide” means a compound having the following formula:

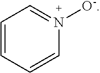

The term “heteroaryloxy”, alone or in combination with other groups, refers to a “heteroaryl” as described herein linked via —O—.

The term “alkylthio” denotes a C₁₋₆-alkyl residue in the sense of the foregoing definition bound via an sulfur atom, for example methylsulfanyl.

The term “carbamoyloxy” means the group —O—CO—NH₂.

The term “C₁₋₆-alkoxy”, alone or in combination with other groups, stands for an —O—C₁₋₆-alkyl radical which may be linear or branched, with single or multiple branching, wherein the alkyl group in general comprises 1 to 6 carbon atoms, for example, methoxy (OMe, MeO), ethoxy (OEt), propoxy, isopropoxy (i-propoxy), n-butoxy, i-butoxy (iso-butoxy), 2-butoxy (sec-butoxy), t-butoxy (tert-butoxy), isopentyloxy (i-pentyloxy) and the like. Particular “C₁₋₆-alkoxy” are groups with 1 to 4 carbon atoms.

The term “halogen-C₁₋₆-alkoxy”, or “C₁₋₆-haloalkoxy”, alone or in combination with other groups, refers to C₁₋₆-alkoxy as defined herein, which is substituted by one or multiple halogens, in particular fluoro. Particular “halogen-C₁₋₆-alkoxy” is fluoro-C₁₋₆-alkoxy.

The term “C₃₋₈₋cycloalkyl” denotes a monovalent saturated monocyclic or bicyclic hydrocarbon group of 3 to 8 ring carbon atoms. Bicyclic means consisting of two saturated carbocycles having one or more carbon atoms in common. Particular C₃₋₈₋cycloalkyl groups are monocyclic. Other particular groups are “C₃₋₆-cycloalkyl” and “C₃₋₄-cycloalkyl” groups. Examples for monocyclic cycloalkyl are cyclopropyl, cyclobutanyl, cyclopentyl, cyclohexyl or cycloheptyl. Examples for bicyclic cycloalkyl are bicyclo[2.2.1]heptanyl, or bicyclo[2.2.2]octanyl. A specific example is cyclopentyl.

The term “heterocycloalkyl” refers to a 3 to 7-membered heterocyclic ring containing at least one heteroatom, such as N, O or S, the number of N atoms being 0, 1, 2 or 3 and the number of O and S atoms each being 0, 1 or 2. The term “5 or 6-membered heterocycloalkyl” refers to a 5 or 6-membered heterocyclic ring as described herein. Examples of heterocyclyl groups include pyrrolidinyl, tetrahydrofuryl, tetrahydrothienyl, tetrahydropyridinyl, tetrahydropyryl, azetidinyl, thiazolidinyl, oxazolidinyl, piperidinyl, morpholinyl, thiomorpholinyl, piperazinyl, azepanyl, diazepanyl, oxazepanyl and the like.

The term “optionally substituted” refers to an Cₐ₋alkyl or C_{b-}alkyl group, which can be unsubstituted or substituted by 1 to 4 substituents individually selected from the group consisting of OH, halogen, cyano, halogen-C₁₋₆-alkoxy and C₁₋₆-alkoxy; or a cycloalkyl group which can be unsubstituted or substituted by 1 to 4 substituents individually selected from the group consisting of OH, halogen, cyano, halogen-C₁₋₆-alkyl, halogen-C₁₋₆-alkoxy and C₁₋₆-alkoxy.

The term “pharmaceutically acceptable salt” refers to salts that are suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid, sulphuric acid, tartaric acid, trifluoroacetic acid and the like. Particular are formic acid, trifluoroacetic acid and hydrochloric acid. Particular are hydrochloric acid, trifluoroacetic acid and fumaric acid.

The terms “pharmaceutically acceptable carrier” and “pharmaceutically acceptable auxiliary substance” refer to carriers and auxiliary substances such as diluents or excipients that are compatible with the other ingredients of the formulation.

The term “prodrug” refers to a structural derivative of a drug which must be chemically transformed within the body into the drug in order to exert its pharmacological or therapeutic action (see Patrick¹⁶ or Ganellin et al.¹⁷).

The term “pharmaceutical composition” encompasses a product comprising specified ingredients in pre-determined amounts or proportions, as well as any product that results, directly or indirectly, from combining specified ingredients in specified amounts. In particular, it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The term “as defined herein” and “as described herein” when referring to a variable incorporates by reference the broad definition of the variable as well as in particular, more particular and most particular definitions, if any.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product. Treatment include prophylactic treatment as well as the acute alleviation of symptoms.

The term “aromatic” denotes the conventional idea of aromaticity as defined in the literature, in particular in IUPAC¹⁸.

The term “pharmaceutically acceptable excipient” denotes any ingredient having no therapeutic activity and being non-toxic such as disintegrators, binders, fillers, solvents, buffers, tonicity agents, stabilizers, antioxidants, surfactants or lubricants used in formulating pharmaceutical products.

The corresponding pharmaceutically acceptable salts with acids can be obtained by standard methods known to the person skilled in the art, e.g. by dissolving the compound of formula I in a suitable solvent such as e.g. dioxan or THF and adding an appropriate amount of the corresponding acid. The products can usually be isolated by filtration or by chromatography. The conversion of a compound of formula (I) or (II) into a pharmaceutically acceptable salt with a base can be carried out by treatment of such a compound with such a base. One possible method to form such a salt is e.g. by addition of 1/n equivalents of a basic salt such as e.g. M(OH)ₙ, wherein M=metal or ammonium cation and n=number of hydroxide anions, to a solution of the compound in a suitable solvent (e.g. ethanol, ethanol-water mixture, tetrahydrofuran-water mixture) and to remove the solvent by evaporation or lyophilisation.

Present invention relates to the use of a mGlu2/3 negative allosteric modulator for the treatment, prevention and/or delay of progression of central nervous system conditions caused by neurodevelopmental defects which result in excessive mGlu2/3 receptor activation in the central nervous system, in particular but not exclusively in cortical regions and hippocampus, and/or that can be corrected by negative allosteric modulation of mGlu2/3 receptor activation.

Present invention relates to the use of a mGlu2 negative allosteric modulator for the treatment, prevention and/or delay of progression of central nervous system conditions caused by neurodevelopmental defects which result in excessive mGlu2 receptor activation in the central nervous system, in particular but not exclusively in cortical regions and hippocampus, and/or that can be corrected by negative allosteric modulation of mGlu2 receptor activation.

Present invention relates to the use of a mGlu3 negative allosteric modulator for the treatment, prevention and/or delay of progression of central nervous system conditions caused by neurodevelopmental defects which result in excessive mGlu3 receptor activation in the central nervous system, in particular but not exclusively in cortical regions and hippocampus, and/or that can be corrected by negative allosteric modulation of mGlu3 receptor activation.

Present invention relates to the use of a mGlu2/3 negative allosteric modulator for the treatment, prevention and/or delay of progression of central nervous system conditions caused by neurodevelopmental defects which result in excessive mGlu2/3 inhibition in the cortex and hippocampus.

A specific aspect of the invention relates to the use as described herein, wherein said central nervous system condition is a disorder of the Autistic Spectrum.

A specific aspect of the invention relates to the use as described herein, wherein said central nervous system condition is autism.

A specific aspect of the invention relates to the use as described herein, wherein said central nervous system condition is Fragile X.

A specific aspect of the invention relates to the use as described herein, wherein the mGlu2/3 negative allosteric modulator is selected from a compound of formula (I) and formula (II),

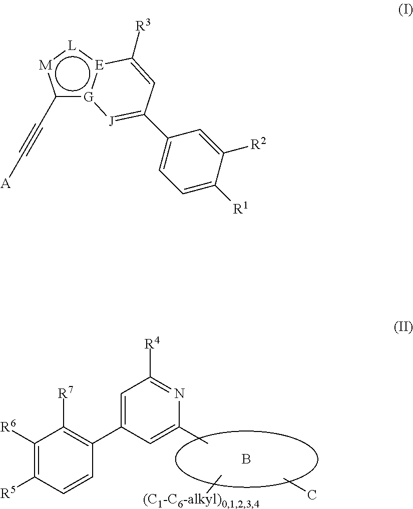

wherein

either E and J are N, G is C and one of L or M is N and the other is CH;

or L and G are N, E is C, and J and M are CH;

or J, G and L are N, E is C and M is CH;

or E and L are N, J and M are CH and G is C;

A is selected from the group consisting of phenyl, pyridin-2-yl, pyridin-3-yl, pyridine-4-yl, pyrimidin-4-yl, pyrimidin-5-yl, pyridazin-2-yl, pyridazin-3-yl, thiazol-2-yl, thiazol-5-yl, and thiophen-2-yl which are optionally substituted by one to four R^{a};

B is selected from the group consisting of imidazolyl, [1,2,4]oxadiazolyl], pyrrolyl, 1H-pyrazolyl, pyridinyl, [1,2,4]triazolyl, thiazolyl, pyrimidinyl and thiophenyl, each of which is optionally substituted by C₁₋₆-alkyl;

C is an optionally substituted aryl or an optionally substituted 5 or 6 membered heteroaryl, wherein the substituents are selected from the group consisting of:

i. halo,

ii. nitro,

iii. C₁₋₆-alkyl optionally substituted by hydroxy,

iv. NR^{aa}R^{bb}, wherein R^{aa} and R^{bb} are independently H, C₁₋₆-alkyl or —(CO)—C₁₋₆-alkyl,

v. —S—C₁₋₆-alkyl,

vi. —(SO₂)—OH,

vii. —(SO₂)—C₁₋₆-alkyl,

viii. —(SO₂)—NR^{cc}R^{dd}, wherein R^{cc} and R^{dd} are independently:
a. H,
b. C₁₋₆-alkyl optionally substituted by hydroxy,
c. C₁₋₆-haloalkyl,
d. C₁₋₆-alkoxy,
e. —(CO)C₁₋₆-alkyl optionally substituted by C₁₋₆-alkoxy,
f. —(CH₂CH₂O)CHR^{ee}, wherein R^{ee} is H or CH₂OH and n is 1, 2, 3, 4, 5, 6, 7, 8, 9 or 10,
g. —(CH₂)ₘ-aryl, wherein m is 1 or 2 and the aryl is optionally substituted by halo or C₁₋₆-alkoxy,
h. —(CH₂)ₚ-C₃₋₆-cycloalkyl, wherein p is 0 or 1,
i. 5 or 6-membered heterocycloalkyl,

ix. —(SO₂)—NR^{ff}R^{gg}, wherein R^{ff} and R^{gg} together with the nitrogen atom to which they are attached form a 4, 5 or 6 membered heterocycloalkyl ring optionally containing a further heteroatom selected from nitrogen, oxygen, sulphur or a SO₂ group, wherein said 4, 5 or 6 membered heterocycloalkyl ring is optionally substituted by a substituent selected from the group consisting of hydroxy, C₁₋₆-alkyl, C₁₋₆-alkoxy which is optionally substituted by hydroxy, and 5 or 6 membered heteroaryloxy,

x. NHSO₂—C₁₋₆-alkyl, and

xi. NHSO₂—NR^{hh}Rⁱⁱ wherein R^{hh} and Rⁱⁱ are independently H, C₁₋₆-alkyl, —(CO)O—C₁₋₆-alkyl, or R^{hh} and Rⁱⁱ together with the nitrogen atom to which they are attached form a 4, 5 or 6 membered heterocycloalkyl ring optionally containing a further heteroatom selected from nitrogen, oxygen or sulphur, wherein said 4, 5 or 6 membered heterocycloalkyl ring is optionally substituted by C₁₋₆-alkyl;

R¹ is H, halo, CF₃, CHF₂, or C₁₋₆-alkyl;

R² is H, halo, C₁₋₆-alkyl, C₁₋₆-alkoxy, CF₃ or CHF₂;

R³ is H, —C(CH₃)₂OH; linear C₁₋₄-alkyl or C₃₋₄-cycloalkyl, which are optionally substituted by one or more substituents selected from the group consisting of 1 to 6 F and 1 to 2 OH;

R⁴ is H, halogen, C₁₋₆-alkyl optionally substituted by hydroxy, C₁₋₆-alkoxy, haloalkyl, C₃₋₆-cycloalkyl;

R⁵ is H, cyano, halogen, C₁₋₆-haloalkyl, C₁₋₆-alkoxy, C₁₋₆-haloalkoxy, C₁₋₆-alkyl or C₃₋₆-cycloalkyl;

R⁶ is halogen, H, C₁₋₆-alkoxy, C₁₋₆-haloalkyl, C₁₋₆-alkyl, C₃₋₆-cycloalkyl, C₁₋₆-haloalkoxy, or is NR^{jj}R^{kk} wherein F^{jj} and R^{kk} are independently selected from the group consisting of: H, C₃₋₈-cycloalkyl, aryl, heteroaryl having from 5 to 12 ring atoms and C₁₋₆-alkyl which optionally substituted by one or more substituent(s) selected from the group consisting of halogen, hydroxy, C₃₋₈-cycloalkyl, aryl, heteroaryl having from 5 to 12 ring atoms and —NR¹¹R^{mm}, wherein R^{ll} and R^{mm} are independently selected from the group consisting of H and C₁₋₆-alkyl;

or R^{jj} and R^{kk} can, together with the nitrogen atom to which they are attached, form an optionally substituted heterocyclic group comprising 5 to 12 ring atoms optionally containing a further heteroatom selected from nitrogen, oxygen or sulphur, wherein said heteroaryl group is optionally substituted by one, two, three, four or five substituents are selected from the group consisting of halogen, hydroxy, C₁₋₆-alkyl and C₁₋₆-haloalkyl;

or R⁵ and R⁶ can together form a dioxo bridge;

R⁷ is H or halo;

R^{a} is halo; hydroxy; cyano; CF₃; NR^{e}R^{f}; C₁₋₆-alkyl optionally substituted by amino or by hydroxy; C₁₋₆-alkoxy; C₃₋₄-cycloalkyl; CO—NR^{b}R^{c}, SO₂—NR^{b}R^{c}; or SO₂—R^{d};

R^{b} and R^{c} may be the same or different and are selected from the group consisting of:

i. H;

ii. straight or branched C₁₋₆-alkyl optionally substituted by one or more substituents selected from the group consisting of:

iii. F, cyano, hydroxy, C₁₋₆-alkoxy, —NH—C(O)—O—C₁₋₆-alkyl, amino, (C₁₋₆-alkyl)amino, di(C₁₋₆-alkyl)amino, C₃₋₆-cycloalkyl, heterocycloalkyl having 5 or 6 ring atoms, aryl or 5 or 6-membered heteroaryl;

iv. C₃₋₆-cycloalkyl;

v. aryl; or

vi. heteroaryl;

or R^{b} and R^{c} may, together with the nitrogen atom to which they are attached, form an heterocyclic ring of 4 to 6 ring members which may be substituted by hydroxy or by C₁₋₆-alkyl;

R^{d} is OH or C₁₋₆-alkyl;

R^{e} and R^{f} are H, C₁₋₆-alkyl optionally substituted by hydroxy, —C(O)—C₁₋₆-alkyl; S(O)₂—C₁₋₆-alkyl;

as well as a pharmaceutically acceptable salt thereof.

A specific aspect of the invention relates to the use as described herein, wherein the mGlu2/3 negative allosteric modulator is selected from a compound of formula (I) and formula (II), as well as prodrugs thereof.

A specific aspect of the invention relates to the use as described herein wherein the mGlu2/3 negative allosteric modulator is selected from a compound of formula (I) and formula (II), wherein

E and J are N, G is C, L is N and M is CH;

A is selected from the group consisting of phenyl, pyridin-2-yl, pyridin-3-yl, pyridine-4-yl, pyrimidin-4-yl, pyrimidin-5-yl, pyridazin-2-yl, pyridazin-3-yl, thiazol-2-yl, thiazol-5-yl, and thiophen-2-yl;

B is selected from the group consisting of imidazolyl, [1,2,4]oxadiazolyl], pyrrolyl, 1H-pyrazolyl, pyridinyl, [1,2,4]triazolyl, thiazolyl, pyrimidinyl and thiophenyl, each of which is optionally substituted by C₁₋₆-alkyl;

C is an optionally substituted aryl, wherein the substituents are selected from the group consisting of:

i. halo,

ii. nitro,

iii. C₁₋₆-alkyl optionally substituted by hydroxy,

iv. NR^{aa}R^{bb}, wherein R^{aa} and R^{bb} are independently H, C₁₋₆-alkyl or —(CO)—C₁₋₆-alkyl,

v. —S—C₁₋₆-alkyl,

vi. —(SO₂)—OH,

vii. —(SO₂)—C₁₋₆-alkyl,

viii. —(SO₂)—NR^{cc}R^{dd}, wherein R^{cc} and R^{dd} are independently:

a. H,

b. C₁₋₆-alkyl optionally substituted by hydroxy,

c. C₁₋₆-haloalkyl,

d. C₁₋₆-alkoxy,

e. —(CO)C₁₋₆-alkyl optionally substituted by C₁₋₆-alkoxy,

R¹ is CF₃;

R² is H;

R³ is linear C₁₋₄-alkyl substituted by one or more substituents selected from the group consisting of 1 to 6 F and 1 to 2 OH;

R⁴ is C₁₋₆-alkyl;

R⁵ is C₁₋₆-haloalkyl;

R⁶ is H;

R⁷ is H;

as well as a pharmaceutically acceptable salt thereof.

A specific aspect of the invention relates to the use as described herein, wherein the mGlu2/3 negative allosteric modulator is selected from a compound of formula (I) and formula (II), wherein

E and J are N, G is C, L is N and M is CH;

A is pyridin-2-yl;

B is pyridinyl,

C is phenyl substituted by SO₂NH₂;

R¹ is CF₃;

R² is H;

R³ is CF₃;

R⁴ i s CH₃;

R⁵ is CF₃;

R⁶ is H;

R⁷ is H;

as well as a pharmaceutically acceptable salt thereof.

A specific aspect of the invention relates to the use as described herein, wherein the mGlu2/3 negative allosteric modulator is a compound of formula (Ia) or a pharmaceutically acceptable salt thereof.

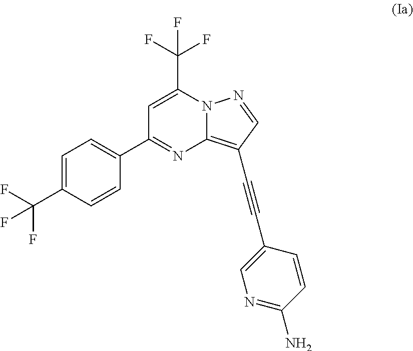 [SMILES: Nc1ccc(C#Cc2cnn3c(C(F)(F)F)cc(-c4ccc(C(F)(F)F)cc4)nc23)cn1]

A specific aspect of the invention relates to the use as described herein, wherein the mGlu2/3 negative allosteric modulator is a compound of formula (Ia) or a prodrug thereof.

A specific aspect of the invention relates to the use as described herein, wherein the mGlu2/3 negative allosteric modulator is a compound of formula (IIa) or (IIb) or a pharmaceutically acceptable salt thereof.

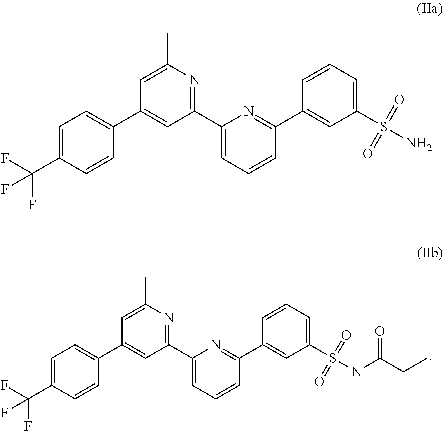 [SMILES: Cc1cc(-c2ccc(C(F)(F)F)cc2)cc(-c2cccc(-c3cccc(S(N)(=O)=O)c3)n2)n1 ; CCC(=O)NS(=O)(=O)c1cccc(-c2cccc(-c3cc(-c4ccc(C(F)(F)F)cc4)cc(C)n3)n2)c1]

A specific aspect of the invention relates to the use as described herein, wherein the mGlu2/3 negative allosteric modulator is a compound of formula (IIa) or a pharmaceutically acceptable salt thereof.

A specific aspect of the invention relates to the use as described herein, wherein the mGlu2/3 negative allosteric modulator is a compound of formula (IIa) or a prodrug thereof.

A specific aspect of the invention relates to the use as described herein, wherein the mGlu2/3 negative allosteric modulator is a compound of formula (IIb) or a pharmaceutically acceptable salt thereof.

A specific aspect of the invention relates to the use as described herein, wherein the mGlu2/3 negative allosteric modulator is a compound of formula (III) or a pharmaceutically acceptable salt thereof.

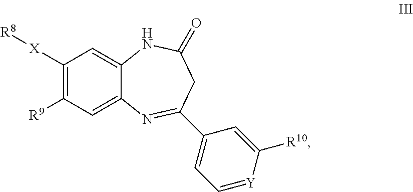

wherein

X is a single bond or an ethynediyl group; and wherein in case X is a single bond,

R⁸ i s hydrogen,
cyano,
halogen,
C₁₋₆-alkyl,
C₁₋₆-alkoxy,
fluoro-C₁₋₆-alkyl,
fluoro-C₁₋₆-alkoxy,
pyrrol-1-yl, or
phenyl, which is unsubstituted or substituted by one or two substituents selected from the group consisting of halogen, C₁₋₆-alkyl or fluoro-C₁₋₆-alkyl;

or in case X is an ethynediyl group,

R⁸ is phenyl, which is unsubstituted or substituted by one or two substituents selected from the group consisting of halogen, C₁₋₆-alkyl or fluoro-C₁₋₆-alkyl;

and wherein

R⁹ is hydrogen,
C₁₋₆-alkyl,
C₂₋₆-alkenyl
C₁₋₆-alkoxy,
halogen,
—NR′R″
pyrrolidin-1-yl,
piperidin-1-yl,
morpholine-4-yl,
fluoro-C₁₋₆-alkyl,
fluoro-C₁₋₆-alkoxy, or
C₁₋₆-alkoxy-(ethoxy), and r is 1, 2, 3 or 4;

R′ is hydrogen, C₁₋₆-alkyl or C₃₋₆-cycloalkyl;
R″ is hydrogen, 1 C₁₋₆-alkyl or C₃₋₆-cycloalkyl;

Y is —CH═ or ═N—;

R¹⁰ is a six-membered aromatic heterocycle containing 1 to 3 nitrogen atoms or a pyridine-N-oxide, which rings are unsubstituted or substituted by one or two substituents selected from the group consisting of
halogen,
fluoro-C₁₋₆-alkyl,
fluoro-C₁₋₆-alkoxy,
cyano,
amino,
C₁₋₆-alkylamino,
C₁₋₆-alkoxy-C₁₋₆-alkylamino,
C₁₋₆-hydroxy-C₁₋₆-alkylamino,
—(CH₂)_{q}—C(O)—OR″,
—(CH₂)_{q}—C(O)—NR′R″,
—(CH₂)_{q}—SO₂—NR′R″,
—(CH₂)_{q}—C(NH₂)═NR″,
hydroxy,
C₁₋₆-alkoxy,
C₁₋₆-alkylthio,
C₃-₆-cycloalkyl, and
C₁₋₆-alkyl, which is optionally substituted by fluoro, —NR′R″, hydroxy, C₁₋₆-alkoxy, pyrrolidin-1-yl, azetidin-1-yl, cyano or carbamoyloxy, whereby R′ and R″ have the meaning specified above; and
q is 0, 1, 2, 3 or 4.

A specific aspect of the invention relates to a method for the treatment, prevention and/or delay of progression of an Autistic Spectrum Disorder in a subject in need of such treatment, which comprises administering to said subject a therapeutically effective amount of a mGlu2/3 negative allosteric modulator as described herein.

A specific aspect of the invention relates to a method for the treatment, prevention and/or delay of progression of autism in a subject in need of such treatment, which comprises administering to said subject a therapeutically effective amount of a mGlu2/3 negative allosteric modulator as described herein.

A specific aspect of the invention relates to a pharmaceutical composition comprising a mGlu2/3 negative allosteric modulator as described herein in a pharmaceutically acceptable form for the treatment, prevention and/or delay of progression of an Autistic Spectrum Disorder.

A specific aspect of the invention relates to a pharmaceutical composition comprising a mGlu2/3 negative allosteric modulator as described herein in a pharmaceutically acceptable form for the treatment, prevention and/or delay of progression of autism.

A specific aspect of the invention relates to a pharmaceutical composition comprising a mGlu2/3 negative allosteric modulator as described herein in a pharmaceutically acceptable form for the treatment, prevention and/or delay of progression of an Autistic Spectrum Disorder.

A specific aspect of the invention relates to a pharmaceutical composition comprising a mGlu2/3 negative allosteric modulator as described herein in a pharmaceutically acceptable form for the treatment, prevention and/or delay of progression of autism.

A specific aspect of the invention relates to a mGlu2/3 negative allosteric modulator as described herein for the treatment, prevention and/or delay of progression of an Autistic Spectrum Disorder.

A specific aspect of the invention relates to a mGlu2/3 negative allosteric modulator as described herein for the treatment, prevention and/or delay of progression of autism.

A specific aspect of the invention relates to a mGlu2/3 negative allosteric modulator as described herein for the preparation of medicaments for the treatment, prevention and/or delay of progression of an Autistic Spectrum Disorder.

A specific aspect of the invention relates to a mGlu2/3 negative allosteric modulator as described herein for the preparation of medicaments for the treatment, prevention and/or delay of progression of autism.

A specific aspect of the invention relates to the use of a mGlu2/3 negative allosteric modulator as described herein for the preparation of medicaments for the treatment, prevention and/or delay of progression of an Autistic Spectrum Disorder.

A specific aspect of the invention relates to the use of a mGlu2/3 negative allosteric modulator as described herein for the preparation of medicaments for the treatment, prevention and/or delay of progression of autism.

### Pharmaceutical Composition

A compound of formula I-III as well as their pharmaceutically acceptable salts can be used as medicaments, e.g. in the form of pharmaceutical preparations. The pharmaceutical preparations can be administered orally, e.g. in the form of tablets, coated tablets, drages, hard and soft gelatin capsules, solutions, emulsions or suspensions. The administration can, however, also be effected rectally, e.g. in the form of suppositories, or parenterally, e.g. in the form of injection solutions.

A compound of formulae I-III and their pharmaceutically acceptable salts can be processed with pharmaceutically inert, inorganic or organic excipients for the production of tablets, coated tablets, drages and hard gelatin capsules. Lactose, corn starch or derivatives thereof, talc, stearic acid or its salts etc. can be used as such excipients e.g. for tablets, drages and hard gelatin capsules. Suitable excipients for soft gelatin capsules are e.g. vegetable oils, waxes, fats, semisolid and liquid polyols etc.

Suitable excipients for the manufacture of solutions and syrups are e.g. water, polyols, saccharose, invert sugar, glucose etc. Suitable excipients for injection solutions are e.g. water, alcohols, polyols, glycerol, vegetable oils etc. Suitable excipients for suppositories are e.g. natural or hardened oils, waxes, fats, semi-liquid or liquid polyols etc.

Moreover, the pharmaceutical preparations can contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary within wide limits and will, of course, be fitted to the individual requirements in each particular case. In general, in the case of oral administration a daily dosage of about 10 to 1000 mg per person of a compound of formulae I-III should be appropriate, although the above upper limit can also be exceeded when necessary.

Examples of compositions according to the invention are, but are not limited to:

### EXAMPLE A

Tablets of the following composition are manufactured in the usual manner:

TABLE 1
possible tablet composition

mg/tablet

| ingredient | 5 | 25 | 100 | 500 |
|---|---|---|---|---|
| Compound of formula I-III | 5 | 25 | 100 | 500 |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### EXAMPLE B-1

Capsules of the following composition are manufactured:

TABLE 2
possible capsule ingredient composition

mg/capsule

| ingredient | 5 | 25 | 100 | 500 |
|---|---|---|---|---|
| Compound of formula I-III | 5 | 25 | 100 | 500 |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

Manufacturing Procedure

1. Mix ingredients 1, 2 and 3 in a suitable mixer for 30 minutes.
2. Add ingredients 4 and 5 and mix for 3 minutes.
3. Fill into a suitable capsule.

A compound of formula I-III, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### EXAMPLE B-2

Soft Gelatin Capsules of the following composition are manufactured:

TABLE 3
possible soft gelatin capsule ingredient composition

| ingredient | mg/capsule |
|---|---|
| Compound of formula I-III | 5 |
| Yellow wax | 8 |
| Hydrogenated Soya bean oil | 8 |
| Partially hydrogenated plant oils | 34 |
| Soya bean oil | 110 |
| Total | 165 |

TABLE 4
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

Manufacturing Procedure

A compound of formula I-III is dissolved in a warm melting of the other ingredients and the mixture is filled into soft gelatin capsules of appropriate size. The filled soft gelatin capsules are treated according to the usual procedures.

### EXAMPLE C

Suppositories of the following composition are manufactured:

TABLE 5
possible suppository composition

| ingredient | mg/supp. |
|---|---|
| Compound of formula I-III | 15 |
| Suppository mass | 1285 |
| Total | 1300 |

Manufacturing Procedure

The suppository mass is melted in a glass or steel vessel, mixed thoroughly and cooled to 45° C. Thereupon, the finely powdered compound of formula I or II is added thereto and stirred until it has dispersed completely. The mixture is poured into suppository moulds of suitable size, left to cool; the suppositories are then removed from the moulds and packed individually in wax paper or metal foil.

### EXAMPLE D

Injection solutions of the following composition are manufactured:

TABLE 6
possible injection solution composition

| ingredient | mg/injection solution. |
|---|---|
| Compound of formula I-III | 3 |
| Polyethylene Glycol 400 | 150 |
| acetic acid | q.s. ad pH 5.0 |
| water for injection solutions | ad 1.0 ml |

Manufacturing Procedure

A compound of formula I-III is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### EXAMPLE E

Sachets of the following composition are manufactured:

TABLE 7
possible sachet composition

| ingredient | mg/sachet |
|---|---|
| Compound of formula I or II | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

Manufacturing Procedure

A compound of formula I-III is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### EXAMPLES

### Example 1

BTBR T+tf/J (BTBR)¹⁹ is an inbred mouse strain demonstrating a robust behavioral phenotype and is known in the art as a model with possible analogies to the diagnostic symptoms of ASD, in particular autism. Deficits in social interactions and social approach, unusual patterns of ultrasonic vocalization, and high levels of repetitive self-grooming are included.²⁰

3-Chambered Social Test

The 3-Chambered Social Test is used to assess autistic-like behaviors. Shortly after a period of habituation a mouse's sociability is determined by evaluating the amount of time the test mouse spends approaching a wire cage (holding cup) containing an unfamiliar mouse.

Procedure

48 male mice BTBR-T+/tfj, 8-9 weeks old, were used in the experiments described herein in 4 groups n=12/group. Further, 6 male stimulus mice (unfamiliar BTBR T+tf/J mice) of similar age and weight were used.

Low light of 20 Lux was used. With the doorways into the two side chambers closed, the test mouse was placed in the middle chamber and allowed to explore the apparatus for 10 min. Thereafter, the doorways were opened and the test mouse was allowed to explore the entire test box for 10 min. The cages were empty. The sociability test was conducted immediately following the habituation phase.

While the test mouse was enclosed in the center compartment of the test box a stimulus mouse was enclosed in a wire cage (holding cup) in one side chamber. The location of the stimulus mouse alternated between the left and right sides of the social test box across subjects. Following placement of the stimulus mouse, the doors were re-opened and the subject mouse was again allowed to explore the entire test box for another 10 minutes. The amount of time spent and the number of entries into each chamber was measured, as was the time spent in a small perimeter around the cup holding the stimulus mouse.

Treatment (3 Hours before Habituation)
Vehicle p.o.—0.3% tween₈₀ in 0.9% NaCl
mglu 2/3 (IIb) 3-10-30 mg/kg p.o.—0.3% tween₈₀ in 0.9% NaCl

Results

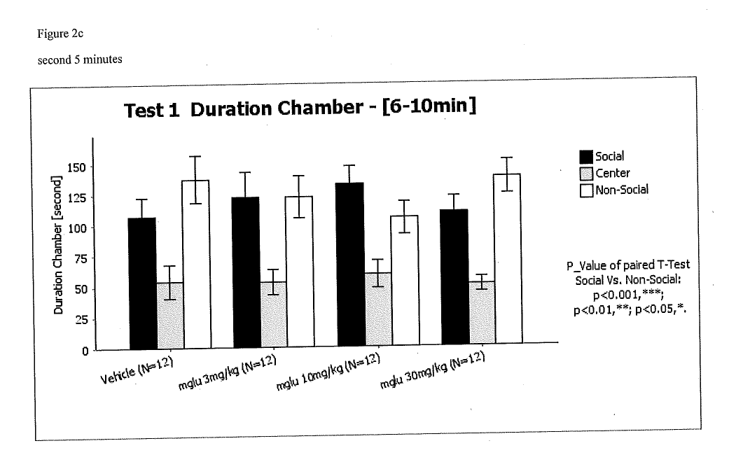

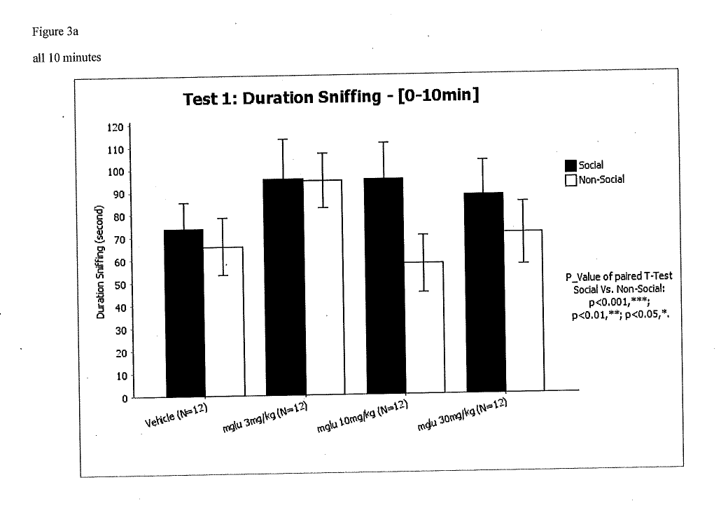

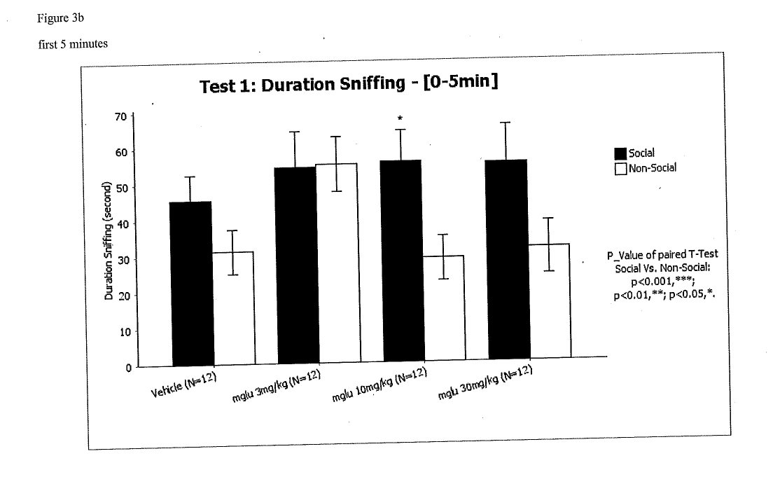

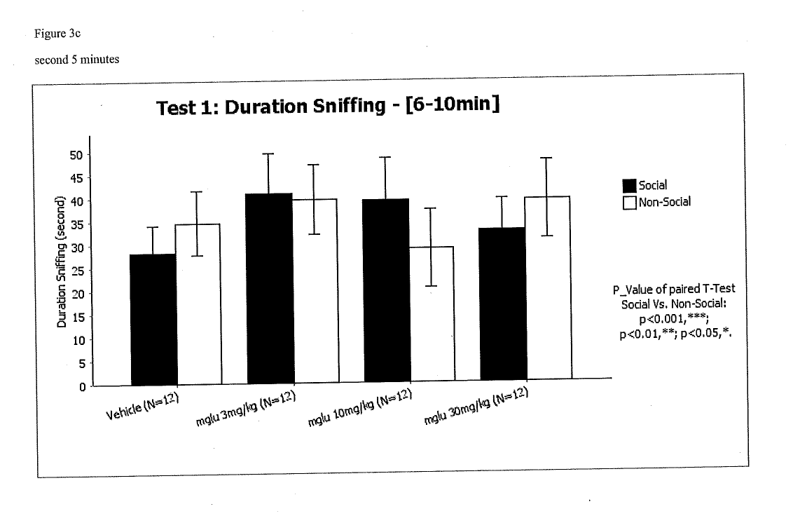

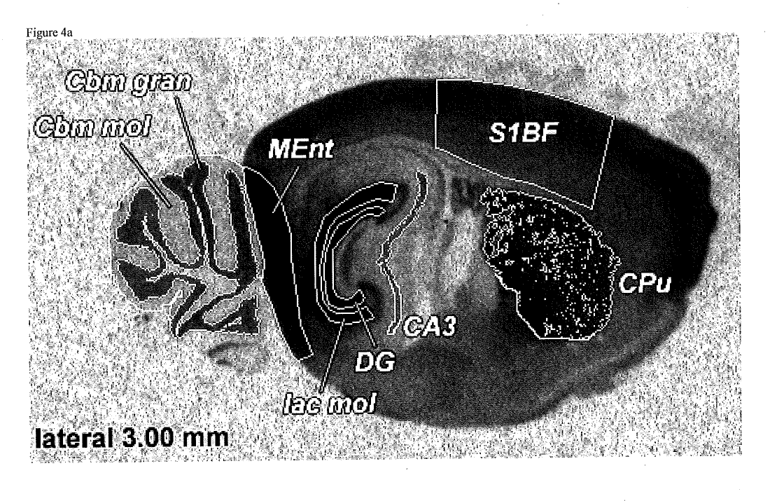

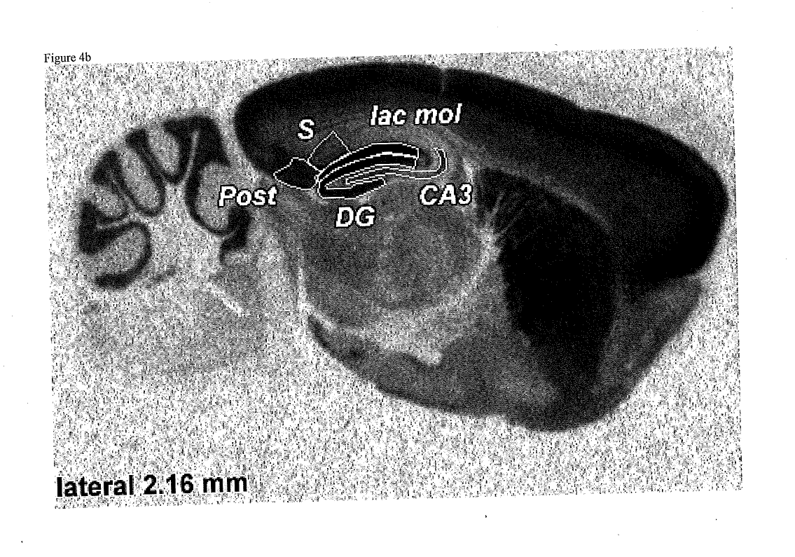

Mice treated with the mglu 2/3 modulator (IIb) showed an increased social preference, especially observed in the first 5 minutes when dosed at 10 mg/kg. (FIGS. 2a, 2b, 2c/3a, 3b, 3c).

### Example 2

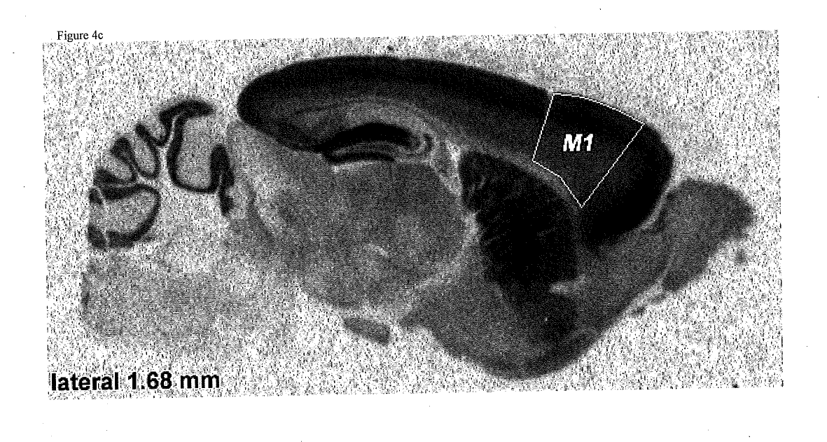

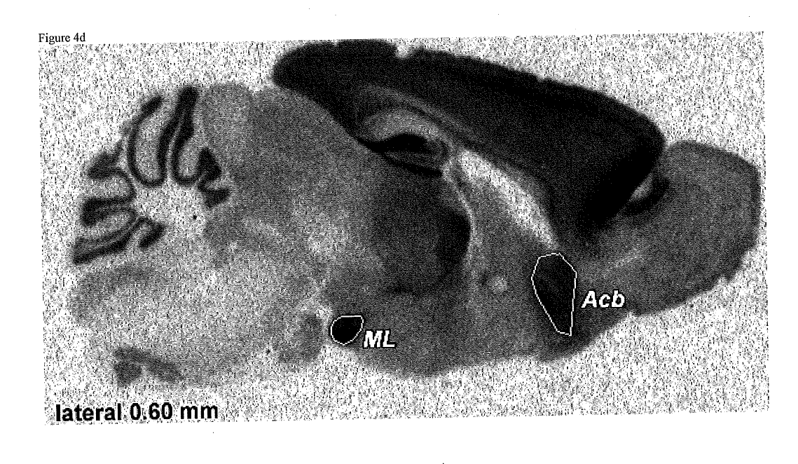

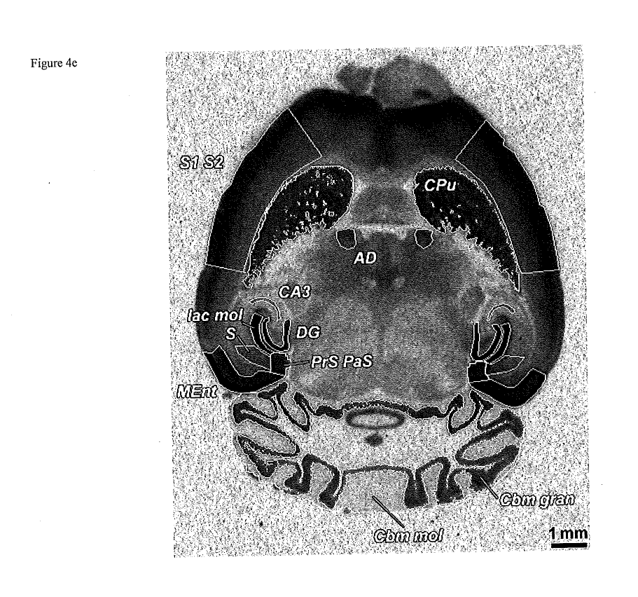

### See FIG. 4a, 4b, 4c, 4d, 4e

[³E1]LY354740 ((+)-2-Aminobicyclo[3.1.0]hexane-2,6-dicarboxylic acid) has been synthesized according to methods known in the art.(Malherbe et al.²¹, Richards et al.²²).

Materials.

[³E1]LY354740 (1 S,2 S,5R,65)-2-aminobicyclo[3.1.0]hexane-2,6-dicarb oxyl ate monohydrate (s.a 35 Ci/mmol) was synthesized at F. Hoffmann-La Roche. The selective group II agonist DCG IV ((2S,2′R,3′R)-2-(2′,3′-dicarboxycyclopropyl)glycine) was synthesized at F.Hoffmann-La Roche. For the cross-sectional study, approximately 3 male C57BL/6J and BTBR mice of similar age (C57Bl/6 mice were 9w (#8661, sagittal), 4m (#6246, saggital) and 16.5m (#8670, horizontal).)

Radioligand Binding to Tissue Sections.

Brains were rapidly dissected from anaesthetized mice (5% fluothane for 30 seconds) and immediately frozen in dry-ice. Parasagittal and horizontal cryostat-cut sections (˜12 μm thick) were mounted on pre-cleaned slides and stored at −20° C. until used. [³E1]LY354740 binding in vitro. For regional distribution studies, sections were pre-incubated at room temperature (22° C.; 10 min) in 50 mM Tris-HCl buffer pH 7.0+EDTA (final volume 130 ml), followed by a further incubation without EDTA and then incubated with 50 nM [³H]LY354740 in the same volume of buffer+2 mM CaCl₂ and MgCl₂ for 60 minutes at 22° C. This was followed by three washes in 130 ml buffer alone at 4° C. (2×30sec.+1 min.; optimal rinse time producing the maximal relative specific binding); non-specific binding was determined in the presence of 10 μM DCG IV ((25,2′R,3′)-2-(2′,3′ -dicarboxycyclopropyl)glycine), a group II metabotropic Glutamate Receptor agonist.

Quantitative Receptor Radioautography.

Radiolabelled sections were exposed, together with tritium microscales (GE Healthcare Life Sciences, UK), to tritium-sensitive imaging plates (BAS-TR2025) for 4 days and subsequently to Hyperfilm Tritium^{R} (GE Healthcare Life Sciences, UK) for 4 weeks at 4° C. The plates were scanned in a Fujifilm BAS-5000 high resolution phosphor imager and measured with an MCID M2 image analysis system (InterFocus Ltd, Haverhill, UK).

Measurements in Sagittal Sections

TABLE 9
Specific binding relative to average of wild-type, %

| | | Lat0.60 | Lat0.60 | Lat1.68 | Lat2.16 | Lat2.16 | Lat2.16 | Lat2.16 | Lat2.16 | Lat3.00 |
|---|---|---|---|---|---|---|---|---|---|---|
| Animal | Parameter | Ac | ML | M1 | CA3 | DG | LMol | Post | S | S1BF |
| BTBR3 | % | 69.4% | 96.1% | 69.4% | 77.7% | 62.8% | 64.9% | 48.7% | 56.6% | 68.9% |
| BTBR6 | | 58.5% | 0.0% | 56.7% | 65.0% | 51.0% | 49.5% | 44.1% | 50.4% | 62.8% |
| Average | | 64.0% | 48.1% | 63.1% | 71.4% | 56.9% | 57.2% | 46.4% | 53.5% | 65.9% |

| | | | | | | Lat3.00 | Lat3.00 | Lat3.00 |
|---|---|---|---|---|---|---|---|---|
| | | Lat3.00 | Lat3.00 | Lat3.00 | Lat3.00 | MEnt + | Cbm | Cbm |
| Animal | Parameter | CPu | LMol | DG | CA3 | Dsc | gran | mol |
| BTBR3 | % | 67.6% | 51.4% | 49.0% | 85.7% | 55.3% | 59.0% | 20.1% |
| BTBR6 | | 58.9% | 50.7% | 50.6% | 79.3% | 50.5% | 72.6% | 18.7% |
| Average | | 63.3% | 51.1% | 49.8% | 82.5% | 52.9% | 65.8% | 19.4% |

Measurements in Horizontal Sections

TABLE 11
Specific binding relative to wild-type, %

| | | hor | | | | | | hor | | | hor | hor |
|---|---|---|---|---|---|---|---|---|---|---|---|---|
| | | S1S2 | hor | hor | hor | hor | hor | PrS | hor | hor | Cbm | Cbm |
| Animal | Parameter | ctx | CPu | AD | LacMol | DG | CA3 | PaS | S | MEnt | gran | mol |
| BTBR1 | % | 65.6% | 66.4% | 86.2% | 50.8% | 53.8% | 105.2% | 54.2% | 51.0% | 47.2% | 50.6% | 60.7% |
| BTBR2 | | 75.1% | 65.3% | 79.7% | 56.2% | 56.7% | 100.7% | 59.7% | 58.4% | 60.1% | 55.1% | 57.1% |
| Average | | 70.3% | 65.9% | 82.9% | 53.5% | 55.2% | 102.9% | 56.9% | 54.7% | 53.7% | 52.8% | 58.9% |

¹ WO 01/29011
² WO 01/29012
³ WO 02/083652
⁴ WO 02/083665
⁵ WO 03/066623
⁶ WO 2005/014002
⁷ WO 2005/040171
⁸ WO 2005/123738
⁹ WO 2006/084634
¹⁰ WO 2006/099972
¹¹ WO 2007/039439
¹² WO 2007/110337
¹³ WO 2008/119689
¹⁴ Genes, Brain and Behavior (2011) 10: 228-235
¹⁵ Curr. Opin. Neurobiol. 19, 231-234 (2009)
¹⁶ G L Patrick, An Introduction to Medicinal Chemistry, Second Edition, pages 239-250
¹⁷ Ganellin and Roberts, Medicinal Chemistry: The role of Organic Chemistry in Drug Research, Second Edition, Academic Press Ltd (1993), Chapter 4
¹⁸ Compendium of Chemical Terminology, 2nd, A. D. McNaught & A. Wilkinson (Eds). Blackwell Scientific Publications, Oxford (1997)
¹⁹ J. L. Silverman*, C. F. Oliver, M. N. Karras, P. T. Gastrell, J. N. Crawley, “AMPAKINE enhancement of social interaction in the BTBR mouse model of autism”, Neuropharmacology 64 (2013) 268-282
²⁰ http://www.psychogenics.com/btbr.html
²¹ Malherbe P, Richards J G, Broger C, Zenner M T, Messer J, Kratzeisen C, Nakanishi S, Mutel

V., J Neurochem. 2005 July;94(1):150-60.
²² Richards G, Messer J, Malherbe P, Pink R, Brockhaus M, Stadler H, Wichmann J, Schaffhauser H, Mutel V., J Comp Neurol. 2005 Jun. 20;487(1):15-27 and Richards G, Messer J, Faull RL, Stadler H, Wichmann J, Huguenin P, Bohrmann B, Mutel V., Brain Res. 2010 Dec. 2;1363:180-90

1. A method of treating, preventing or delaying the progression of a central nervous system condition in a subject, wherein said condition is caused by neurodevelopmental defects which result in excessive mGlu2/3 receptor activation in the central nervous system and/or that can be corrected by negative allosteric modulation of mGlu2/3 receptor activation, comprising administering a therapeutically effective amount of an mGlu2/3 negative allosteric modulator, or a pharmaceutically acceptable salt thereof, to a subject in need thereof.

2. The method of claim 1, wherein said central nervous system condition is caused by neurodevelopmental defects which result in excessive mGlu2/3 inhibition in the cortex and hippocampus.

3. The method of claim 1, wherein said central nervous system condition is a disorder of the Autistic Spectrum.

4. The method of claim 1, wherein said central nervous system condition is autism.

5. The method of claim 1, wherein said mGlu2/3 negative allosteric modulator is selected from a compound of formula (I) and formula (II) 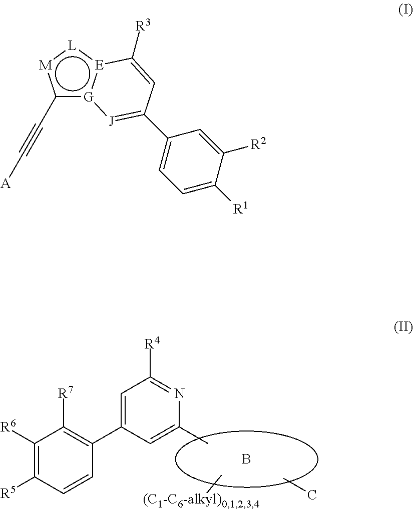
wherein
either E and J are N, G is C and one of L or M is N and the other is CH;
or L and G are N, E is C, and J and M are CH;
or J, G and L are N, E is C and M is CH;
or E and L are N, J and M are CH and G is C;
A is selected from the group consisting of phenyl, pyridin-2-yl, pyridin-3-yl, pyridine-4-yl, pyrimidin-4-yl, pyrimidin-5-yl, pyridazin-2-yl, pyridazin-3-yl, thiazol-2-yl, thiazol-5-yl, and thiophen-2-yl which are optionally substituted by one to four R^{a};
B is selected from the group consisting of imidazolyl, [1,2,4]oxadiazolyl], pyrrolyl, 1H-pyrazolyl, pyridinyl, [1,2,4]triazolyl, thiazolyl, pyrimidinyl and thiophenyl, each of which is optionally substituted by C₁₋₆-alkyl;
C is an optionally substituted aryl or an optionally substituted 5 or 6 membered heteroaryl, wherein the substituents are selected from the group consisting of:
i. halo,
ii. nitro,
iii. C₁₋₆-alkyl optionally substituted by hydroxy,
iv. NR^{aa}R^{bb}, wherein R^{aa} and R^{bb} are independently H, C₁₋₆-alkyl or —(CO)—C₁₋₆-alkyl,
v. —S—C₁₋₆-alkyl,
vi. —(SO₂)—OH,
vii. —(SO₂)—C₁₋₆-alkyl,
viii. —(SO₂)—NR^{cc}R^{dd}, wherein R^{cc} and R^{dd} are independently:
a. H,
b. C₁₋₆-alkyl optionally substituted by hydroxy,
c. C₁₋₆-haloalkyl,
d. C₁₋₆-alkoxy,
e. —(CO)C₁₋₆-alkyl optionally substituted by C₁₋₆-alkoxy,
f. —(CH₂CH₂O)ₙCHR^{ee}, wherein R^{ee} is H or CH₂OH and n is 1, 2, 3, 4, 5, 6, 7, 8, 9 or 10,
g. —(CH₂)ₘ-aryl, wherein m is 1 or 2 and the aryl is optionally substituted by halo or C₁₋₆-alkoxy,
h. —(CH₂)ₚ-C₃₋₆-cycloalkyl, wherein p is 0 or 1,
i. 5 or 6-membered heterocycloalkyl,
ix. —(SO₂)—NR^{ff}R^{gg}, wherein R^{ff} and R^{gg} together with the nitrogen atom to which they are attached form a 4, 5 or 6 membered heterocycloalkyl ring optionally containing a further heteroatom selected from nitrogen, oxygen, sulphur or a SO₂ group, wherein said 4, 5 or 6 membered heterocycloalkyl ring is optionally substituted by a substituent selected from the group consisting of hydroxy, C₁₋₆-alkyl, C₁₋₆-alkoxy which is optionally substituted by hydroxy, and 5 or 6 membered heteroaryloxy,
x. NHSO₂—C₁₋₆-alkyl, and
xi. NHSO₂—NR^{hh}Rⁱⁱ wherein R^{hh} and Rⁱⁱ are independently H, C₁₋₆-alkyl, —(CO)O—C₁₋₆-alkyl, or R^{hh} and Rⁱⁱ together with the nitrogen atom to which they are attached form a 4, 5 or 6 membered heterocycloalkyl ring optionally containing a further heteroatom selected from nitrogen, oxygen and sulphur, wherein said 4, 5 or 6 membered heterocycloalkyl ring is optionally substituted by C₁₋₆-alkyl;
R¹ is H, halo, CF₃, CHF₂, or C₁₋₆-alkyl;
R² is H, halo, C₁₋₆-alkyl, C₁₋₆-alkoxy, CF₃ or CHF₂;
R³ is H, —C(CH₃)₂OH; linear C₁₋₄-alkyl or C₃₋₄-cycloalkyl, which are optionally substituted by one or more substituents selected from the group consisting of 1 to 6 F and 1 to 2 OH;
R⁴ is H, halogen, C₁₋₆-alkyl optionally substituted by hydroxy, C₁₋₆-alkoxy, C₁₋₆-haloalkyl, or C₃₋₆-cycloalkyl;
R⁵ is H, cyano, halogen, C₁₋₆-haloalkyl, C₁₋₆-alkoxy, C₁₋₆-haloalkoxy, C₁₋₆-alkyl or C₃₋₆-cycloalkyl;
R⁶ is halogen, H, C₁₋₆-alkoxy, C₁₋₆-haloalkyl, C₁₋₆-alkyl, C₃₋₆-cycloalkyl, C₁₋₆-haloalkoxy, or is NR^{jj}R^{kk} wherein R^{jj} and R^{kk} are independently selected from the group consisting of: H, C₃₋₈-cycloalkyl, aryl, heteroaryl having from 5 to 12 ring atoms and C₁₋₆-alkyl which optionally substituted by one or more substituent(s) selected from the group consisting of halogen, hydroxy, C₃₋₈-cycloalkyl, aryl, heteroaryl having from 5 to 12 ring atoms and —NR^{ll}R^{mm}, wherein R^{ll} and R^{mm} are independently selected from the group consisting of H and C₁₋₆-alkyl;
or R^{jj} and R^{kk} can, together with the nitrogen atom to which they are attached, form an optionally substituted heterocyclic group comprising 5 to 12 ring atoms optionally containing a further heteroatom selected from nitrogen, oxygen or sulphur, wherein said heteroaryl group is optionally substituted by one, two, three, four or five substituents are selected from the group consisting of halogen, hydroxy, C₁₋₆-alkyl and C₁₋₆-haloalkyl;
or R⁵ and R⁶ can together form a dioxo bridge;
R⁷ is H or halo;
R^{a} is halo; hydroxy; cyano; CF₃; NR^{e}R^{f}; C₁₋₆-alkyl optionally substituted by amino or by hydroxy; C₁₋₆-alkoxy; C₃₋₄-cycloalkyl; CO—NR^{b}R^{c}, SO₂—NR^{b}R^{c}; or SO₂—R^{d};
R^{b} and R^{c} may be the same or different and are selected from the group consisting of:
i. H;
ii. straight or branched C₁₋₆-alkyl optionally substituted by one or more substituents selected from the group consisting of:
iii. F, cyano, hydroxy, C₁₋₆-alkoxy, —NH—C(O)—O—C₁₋₆-alkyl, amino, (C₁₋₆-alkyl)amino, di(C₁₋₆-alkyl)amino, C₃₋₆-cycloalkyl, heterocycloalkyl having 5 or 6 ring atoms, aryl or 5 or 6-memb ered heteroaryl;
iv. C₃₋₆-cycloalkyl;
v. aryl; or
vi. heteroaryl;
or R^{b} and R^{c} may, together with the nitrogen atom to which they are attached, form an heterocyclic ring of 4 to 6 ring members which may be substituted by hydroxy or by C₁₋₆-alkyl;
R^{d} is OH or C₁₋₆-alkyl;
R^{e} and R^{f} are H, C₁₋₆-alkyl optionally substituted by hydroxy, —C(O)—C₁₋₆-alkyl; S(O)₂—C₁₋₆-alkyl;
or a pharmaceutically acceptable salt thereof.

6. The method of claim 1, wherein said mGlu2/3 negative allosteric modulator is selected from a compound of formula (I) and formula (II), wherein
E and J are N, G is C, L is N and M is CH;
A is selected from the group consisting of phenyl, pyridin-2-yl, pyridin-3-yl, pyridine-4-yl, pyrimidin-4-yl, pyrimidin-5-yl, pyridazin-2-yl, pyridazin-3-yl, thiazol-2-yl, thiazol-5-yl, and thiophen-2-yl;
B is selected from the group consisting of imidazolyl, [1,2,4]oxadiazolyl], pyrrolyl, 1H-pyrazolyl, pyridinyl, [1,2,4]triazolyl, thiazolyl, pyrimidinyl and thiophenyl, each of which is optionally substituted by C₁₋₆-alkyl;
C is an optionally substituted aryl, wherein the substituents are selected from the group consisting of:
i. halo,
ii. nitro,
iii. C₁₋₆-alkyl optionally substituted by hydroxy,
iv. NR^{aa}R^{bb}, wherein R^{aa} and R^{bb} are independently H, C₁₋₆-alkyl or —(CO)—C₁₋₆-alkyl,
v. —S—C₁₋₆-alkyl,
vi. —(SO₂)—OH,
vii. —(SO₂)—C₁₋₆-alkyl,
viii. —(SO₂)—NR^{cc}R^{dd}, wherein R^{cc} and R^{dd} are independently:
a. H,
b. C₁₋₆-alkyl optionally substituted by hydroxy,
c. C₁₋₆-haloalkyl,
d. C₁₋₆-alkoxy,
e. —(CO)C₁₋₆-alkyl optionally substituted by C₁₋₆-alkoxy,
R¹ is CF₃;
R² is H;
R³ is linear C₁₋₄-alkyl substituted by one or more substituents selected from the group consisting of 1 to 6 F and 1 to 2 OH;
R⁴ is C₁₋₆-alkyl;
R⁵ is C₁₋₆-haloalkyl;
R⁶ is H;
R⁷ is H;
or a pharmaceutically acceptable salt thereof.

7. The method of claim 1, wherein said mGlu2/3 negative allosteric modulator is selected from a compound of formula (I) and formula (II), wherein
E and J are N, G is C, L is N and M is CH;
A is pyridin-2-yl;
B is pyridinyl,
C is phenyl substituted by SO₂NH₂;
R¹ is CF₃;
R² is H;
R³ is CF₃;
R⁴ is CH₃;
R⁵ is CF₃;
R⁶ is H;
R⁷ is H;
or a pharmaceutically acceptable salt thereof.

8. The method of claim 1, wherein the mGlu2/3 negative allosteric modulator is a compound of formula (Ia) or a pharmaceutically acceptable salt thereof

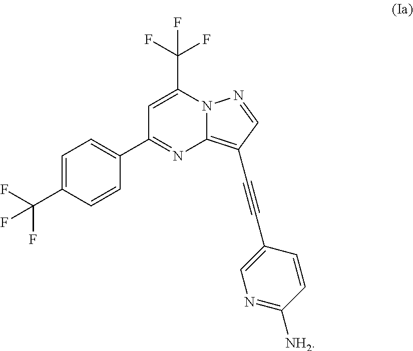 [SMILES: Nc1ccc(C#Cc2cnn3c(C(F)(F)F)cc(-c4ccc(C(F)(F)F)cc4)nc23)cn1]

9. The method of claim 1, wherein the mGlu2/3 negative allosteric modulator is a compound of formula (IIa) or (IIb), or a pharmaceutically acceptable salt thereof

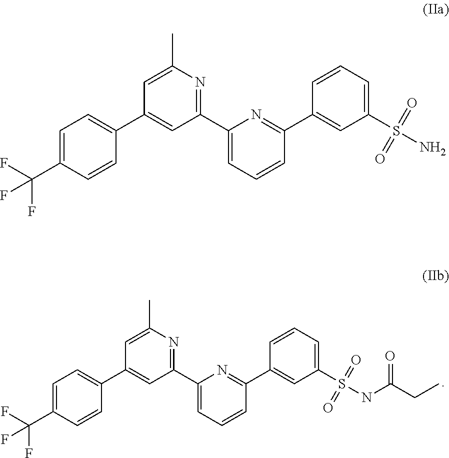 [SMILES: Cc1cc(-c2ccc(C(F)(F)F)cc2)cc(-c2cccc(-c3cccc(S(N)(=O)=O)c3)n2)n1 ; CCC(=O)NS(=O)(=O)c1cccc(-c2cccc(-c3cc(-c4ccc(C(F)(F)F)cc4)cc(C)n3)n2)c1]

10. A method for the treatment, prevention and/or delay of progression of an Autistic Spectrum Disorder in a subject in need of such treatment, which comprises administering to said subject a therapeutically effective amount of an mGlu2/3 negative allosteric modulator, or a pharmaceutically acceptable salt thereof.

11. The method of claim 10, wherein said mG 2/3 negative allosteric modulator is selected from a compound of formula (I) and formula (II) 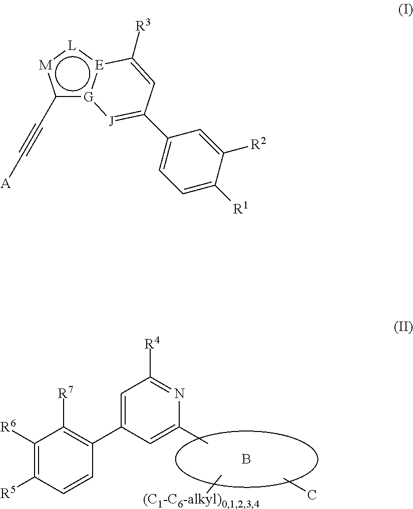
wherein
either E and J are N, G is C and one of L or M is N and the other is CH;
or L and G are N, E is C, and J and M are CH;
or J, G and L are N, E is C and M is CH;
or E and L are N, J and M are CH and G is C;
A is selected from the group consisting of phenyl, pyridin-2-yl, pyridin-3-yl, pyridine-4-yl, pyrimidin-4-yl, pyrimidin-5-yl, pyridazin-2-yl, pyridazin-3-yl, thiazol-2-yl, thiazol-5-yl, and thiophen-2-yl which are optionally substituted by one to four R^{a};
B is selected from the group consisting of imidazolyl, [1,2,4]oxadiazolyl], pyrrolyl, 1H-pyrazolyl, pyridinyl, [1,2,4]triazolyl, thiazolyl, pyrimidinyl and thiophenyl, each of which is optionally substituted by C₁₋₆-alkyl;
C is an optionally substituted aryl or an optionally substituted 5 or 6 membered heteroaryl, wherein the substituents are selected from the group consisting of:
xii. halo,
xiii. nitro,
xiv. C₁₋₆-alkyl optionally substituted by hydroxy,
xv. NR^{aa}R^{bb} wherein R^{aa} and R^{bb} are independently H, C₁₋₆-alkyl or —(CO)—C₁₋₆-alkyl,
xvi. —S—C₁₋₆-alkyl,
xvii. —(SO₂)—OH,
xviii. (SO₂)—C₁₋₆-alkyl,
xix. —(SO₂)—NR^{cc}R^{dd}, wherein R^{cc} and R^{dd} are independently:
j. H,
k. C₁₋₆-alkyl optionally substituted by hydroxy,
l. C₁₋₆-haloalkyl,
m. C₁₋₆-alkoxy,
n. —(CO)C₁₋₆-alkyl optionally substituted by C₁₋₆-alkoxy,
o. —(CH₂CH₂O)ₙCHR^{ee}, wherein R^{ee} is H or CH₂OH and n is 1, 2, 3, 4, 5, 6, 7, 8, 9 or 10,
p. —(CH₂)ₘ-aryl, wherein m is 1 or 2 and the aryl is optionally substituted by halo or C₁₋₆-alkoxy,
q. —(CH₂)ₚ-C₃₋₆-cycloalkyl, wherein p is 0 or 1,
r. 5 or 6-membered heterocycloalkyl,
xx. —(SO₂)-NR^{ff}N^{gg}, wherein R^{ff} and R^{gg} together with the nitrogen atom to which they are attached form a 4, 5 or 6 membered heterocycloalkyl ring optionally containing a further heteroatom selected from nitrogen, oxygen, sulphur or a SO₂ group, wherein said 4, 5 or 6 membered heterocycloalkyl ring is optionally substituted by a substituent selected from the group consisting of hydroxy, C₁₋₆-alkoxy which is optionally substituted by hydroxy, and 5 or 6 membered heteroaryloxy,
xxi. NHSO₂—C₁₋₆-alkyl, and
xxii. NHSO₂—NR^{hh}Rⁱⁱ wherein R^{hh} and Rⁱⁱ are independently H, C₁₋₆-alkyl, —(CO)O—C₁₋₆-alkyl, or R^{hh} and R^{ll} together with the nitrogen atom to which they are attached form a 4, 5 or 6 membered heterocycloalkyl ring optionally containing a further heteroatom selected from nitrogen, oxygen and sulphur, wherein said 4, 5 or 6 membered heterocycloalkyl ring is optionally substituted by C₁₋₆-alkyl;
R¹ is H, halo, CF₃, CHF₂, or C₁₋₆-alkyl;
R² is H, halo, C₁₋₆-alkyl, C₁₋₆-alkoxy, CF₃ or CHF₂;
R³ is H, —C(CH₃)₂OH; linear C₁₋₄-alkyl or C₃₋₄-cycloalkyl, which are optionally substituted by one or more substituents selected from the group consisting of 1 to 6 F and 1 to 2 OH;
R⁴ is H, halogen, C₁₋₆-alkyl optionally substituted by hydroxy, C₁₋₆-alkoxy, haloalkyl, or C₃₋₆-cycloalkyl;
R⁵ is H, cyano, halogen, C₁₋₆-haloalkyl, C₁₋₆-alkoxy, C₁₋₆-haloalkoxy, C₁₋₆-alkyl or C₃₋₆-cycloalkyl;
R⁶ is halogen, H, C₁₋₆-alkoxy, C₁₋₆-haloalkyl, C₁₋₆-alkyl, C₃₋₆-cycloalkyl, C₁₋₆-haloalkoxy, or is NR^{jj}R^{kk} wherein R^{jj} and R^{kk} are independently selected from the group consisting of: H, C₃₋₈-cycloalkyl, aryl, heteroaryl having from 5 to 12 ring atoms and C₁₋₆-alkyl which optionally substituted by one or more substituent(s) selected from the group consisting of halogen, hydroxy, C₃₋₈-cycloalkyl, aryl, heteroaryl having from 5 to 12 ring atoms and —NR¹¹R^{mm}, wherein R^{ll} and R^{mm} are independently selected from the group consisting of H and C₁₋₆-alkyl;
or R^{jj} and R^{kk} can, together with the nitrogen atom to which they are attached, form an optionally substituted heterocyclic group comprising 5 to 12 ring atoms optionally containing a further heteroatom selected from nitrogen, oxygen or sulphur, wherein said heteroaryl group is optionally substituted by one, two, three, four or five substituents are selected from the group consisting of halogen, hydroxy, C₁₋₆-alkyl and C₁₋₆-haloalkyl;
or R⁵ and R⁶ can together form a dioxo bridge;
R⁷ is H or halo;
R^{a} is halo; hydroxy; cyano; CF₃; NR^{c}R^{f}; C₁₋₆-alkyl optionally substituted by amino or by hydroxy; C₁₋₆-alkoxy; C₃₋₄-cycloalkyl; CO—NR^{b}R^{c}, SO₂—NR^{b}R^{c}; or SO₂—R^{d};
R^{b} and R^{c} may be the same or different and are selected from the group consisting of:
vii. H;
viii. straight or branched C₁₋₆-alkyl optionally substituted by one or more substituents selected from the group consisting of:
ix. F, cyano, hydroxy, C₁₋₆-alkoxy, —NH—C(O)—O—C₁₋₆-alkyl, amino, (C₁₋₆-alkyl)amino, di(C₁₋₆-alkyl)amino, C₃₋₆-cycloalkyl, heterocycloalkyl having 5 or 6 ring atoms, aryl or 5 or 6-membered heteroaryl;
x. C₃₋₆-cycloalkyl;
xi. aryl; or
xii. heteroaryl;
or R^{b} and R^{c} may, together with the nitrogen atom to which they are attached, form an heterocyclic ring of 4 to 6 ring members which may be substituted by hydroxy or by C₁₋₆-alkyl;
R^{d} is OH or C₁₋₆-alkyl;
R^{e} and R^{f} are H, C₁₋₆-alkyl optionally substituted by hydroxy, —C(O)—C₁₋₆-alkyl; S(O)₂-C₁₋₆-alkyl;
or a pharmaceutically acceptable salt thereof.

12. A pharmaceutical composition comprising a mGlu2/3 negative allosteric modulator in a pharmaceutically acceptable form for the treatment, prevention and/or delay of progression of an Autistic Spectrum Disorder.

13. The composition of claim 12, wherein said mGlu2/3 negative allosteric modulator is selected from a compound of formula (I) and formula (II) 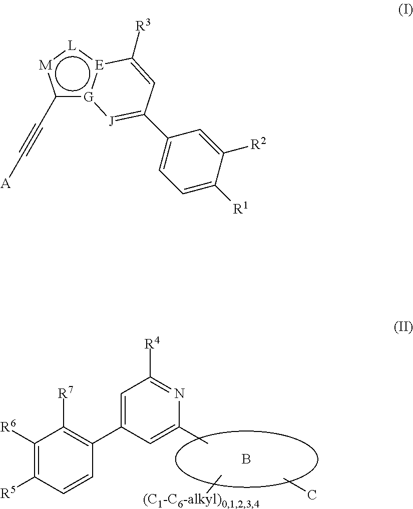
wherein
either E and J are N, G is C and one of L or M is N and the other is CH;
or L and G are N, E is C, and J and M are CH;
or J, G and L are N, E is C and M is CH;
or E and L are N, J and M are CH and G is C;
A is selected from the group consisting of phenyl, pyridin-2-yl, pyridin-3-yl, pyridine-4-yl, pyrimidin-4-yl, pyrimidin-5-yl, pyridazin-2-yl, pyridazin-3-yl, thiazol-2-yl, thiazol-5-yl, and thiophen-2-yl which are optionally substituted by one to four R^{a};
B is selected from the group consisting of imidazolyl, [1,2,4]oxadiazolyl], pyrrolyl, 1H-pyrazolyl, pyridinyl, [1,2,4]triazolyl, thiazolyl, pyrimidinyl and thiophenyl, each of which is optionally substituted by C₁₋₆-alkyl;
C is an optionally substituted aryl or an optionally substituted 5 or 6 membered heteroaryl, wherein the substituents are selected from the group consisting of:
xxiii. halo,
xxiv. nitro,
xxv. C₁₋₆-alkyl optionally substituted by hydroxy,
xxvi. NR^{aa}R^{bb}, wherein R^{aa} and R^{bb} are independently H, C₁₋₆-alkyl or —(CO)—C₁₋₆-alkyl,
xxvii. —S—C₁₋₆-alkyl,
xxviii. —(SO₂)—OH,
xxix. —(SO₂)—C₁₋₆-alkyl,
xxx. —(SO₂)—NR^{cc}R^{dd}, wherein R^{cc} and R^{dd} are independently:
s. H,
t. C₁₋₆-alkyl optionally substituted by hydroxy,
u. C₁₋₆-haloalkyl,
v. C₁₋₆-alkoxy,
w. —(CO)C₁₋₆-alkyl optionally substituted by C₁₋₆-alkoxy,
x. —(CH₂CH₂O)ₙCHR^{ee}, wherein R^{ee} is H or CH₂OH and n is 1, 2, 3, 4, 5, 6, 7, 8, 9 or 10,
y. —(CH₂)ₘ-aryl, wherein m is 1 or 2 and the aryl is optionally substituted by halo or C₁₋₆-alkoxy,
z. —(CH₂)ₚ—C₃₋₆-cycloalkyl, wherein p is 0 or 1,
aa. 5 or 6-membered heterocycloalkyl,
xxxi. —(SO₂)—N^{ff}R^{gg}, wherein R^{ff} and R^{gg} together with the nitrogen atom to which they are attached form a 4, 5 or 6 membered heterocycloalkyl ring optionally containing a further heteroatom selected from nitrogen, oxygen, sulphur or a SO₂ group, wherein said 4, 5 or 6 membered heterocycloalkyl ring is optionally substituted by a substituent selected from the group consisting of hydroxy, C₁₋₆-alkoxy which is optionally substituted by hydroxy, and 5 or 6 membered heteroaryloxy,
xxxii. NHSO₂—C₁₋₆-alkyl, and
xxxiii. NHSO₂—NR^{hh}Rⁱⁱ wherein R^{hh} and Rⁱⁱ are independently H, —(CO)O—C₁₋₆-alkyl, or R^{hh} and Rⁱⁱ together with the nitrogen atom to which they are attached form a 4, 5 or 6 membered heterocycloalkyl ring optionally containing a further heteroatom selected from nitrogen, oxygen and sulphur, wherein said 4, 5 or 6 membered heterocycloalkyl ring is optionally substituted by C₁₋₆-alkyl;
R¹ is H, halo, CF₃, CHF₂, or C₁₋₆-alkyl;
R² is H, halo, C₁₋₆-alkyl, C₁₋₆-alkoxy, CF₃ or CHF₂;
R³ is H, —C(CH₃)₂OH; linear C₁₋₄-alkyl or C₃₋₄-cycloalkyl, which are optionally substituted by one or more substituents selected from the group consisting of 1 to 6 F and 1 to 2 OH;
R⁴ is H, halogen, C₁₋₆-alkyl optionally substituted by hydroxy, C₁₋₆-alkoxy, C₁₋₆-haloalkyl, or C₃₋₆-cycloalkyl;
R⁵ is H, cyano, halogen, C₁₋₆-haloalkyl, C₁₋₆-alkoxy, C₁₋₆-haloalkoxy, C₁₋₆-alkyl or C₃₋₆-cycloalkyl;
R⁶ is halogen, H, C₁₋₆-alkoxy, C₁₋₆-haloalkyl, C₃₋₆-cycloalkyl, C₁₋₆-haloalkoxy, or is NR^{jj}R^{kk} wherein R^{jj} and R^{kk} are independently selected from the group consisting of: H, C₃₋₈-cycloalkyl, aryl, heteroaryl having from 5 to 12 ring atoms and C₁₋₆-alkyl which optionally substituted by one or more substituent(s) selected from the group consisting of halogen, hydroxy, C₃₋₈-cycloalkyl, aryl, heteroaryl having from 5 to 12 ring atoms and —NR^{ll}R^{mm}, wherein R^{ll} and R^{mm} are independently selected from the group consisting of H and C₁₋₆-alkyl;
or R^{jj} and R^{kk} can, together with the nitrogen atom to which they are attached, form an optionally substituted heterocyclic group comprising 5 to 12 ring atoms optionally containing a further heteroatom selected from nitrogen, oxygen or sulphur, wherein said heteroaryl group is optionally substituted by one, two, three, four or five substituents are selected from the group consisting of halogen, hydroxy, C₁₋₆-alkyl and C₁₋₆-haloalkyl;
or R⁵ and R⁶ can together form a dioxo bridge;
R⁷ is H or halo;
R^{a} is halo; hydroxy; cyano; CF₃; NR^{e}R^{f}; C₁₋₆-alkyl optionally substituted by amino or by hydroxy; C₁₋₆-alkoxy; C₃₋₄-cycloalkyl; CO—NR^{b}R^{c}, SO₂—NR^{b}R^{c}; or SO₂—R^{d};
R^{b} and R^{c} may be the same or different and are selected from the group consisting of:
xiii. H;
xiv. straight or branched C₁₋₆-alkyl optionally substituted by one or more substituents selected from the group consisting of:
xv. F, cyano, hydroxy, C₁₋₆-alkoxy, —NH—C(O)—O—C₁₋₆-alkyl, amino, (C₁₋₆-alkyl)amino, di(C₁₋₆-alkyl)amino, C₃₋₆-cycloalkyl, heterocycloalkyl having 5 or 6 ring atoms, aryl or 5 or 6-membered heteroaryl;
xvi. C₃₋₆-cycloalkyl;
xvii. aryl; or
xviii. heteroaryl;
or R^{b} and R^{c} may, together with the nitrogen atom to which they are attached, form an heterocyclic ring of 4 to 6 ring members which may be substituted by hydroxy or by C₁₋₆-alkyl;
R^{d} is OH or C₁₋₆-alkyl;
R^{e} and R^{f} are H, C₁₋₆-alkyl optionally substituted by hydroxy, —C(O)—C₁₋₆-alkyl; S(O)₂—C₁₋₆-alkyl;
or a pharmaceutically acceptable salt thereof.
